(19) United States
(12) Patent Application Publication
(10) Pub. No.: US 2026/0345065 A1
(43) Pub. Date: Nov. 5, 2026

(54) SYSTEM AND METHOD FOR DETERMINING THE RISK OF FETAL HYPOXIA

(71) Applicants: UNIVERSITE PARIS CITE, PARIS (FR); GENOS CARE, BOIS-COLOMBES (FR); ASSISTANCE PUBLIQUE HOPI TAUX DE PARIS, PARIS (FR); UNIV PARIS XIII PARIS-NORD VILLE TANEUSE, VILLETANEUSE (FR)

(72) Inventors: Imane BEN M'BAREK, BOIS-COLOMBES (FR); Grégoire JAUVION, BOIS-COLOMBES (FR); Pierre-François CECCALDI, PARIS (FR)

(21) Appl. No.: 18/997,322

(22) PCT Filed: Jul. 6, 2023
(86) PCT No.: PCT/FR2023/000129

§ 371 Date: Jan. 21, 2025

(30) Foreign Application Priority Data
Jul. 23, 2022 (FR) FR2207583

(51) Int. Cl. A61B 5/00; A61B 5/024; G16H 50/30

(52) U.S. Cl. CPC A61B 5/4362; A61B 5/02405; A61B 5/02411; A61B 5/4356; A61B 5/7267; A61B 5/7275; G16H 50/30

(57) ABSTRACT

A system for providing a method for the automated analysis of cardiotocographic data, in order to measure the risk of fetal hypoxia, of between 0% for absence of any risk and 100% for confirmed fetal hypoxia. The system according to the invention makes it possible, by means of the artificial intelligence model facilitated by the neural network and by means of the mathematical regression model, to determine the contributions of the various risk indicators capable of being subsequently interpreted by practitioners. The models for assessing the risk of fetal hypoxia, determined by means of a neural network, and the contributions of the various indicators used to determine the risk of fetal hypoxia, determined by logistic regression, are trained on a retrospective database.

### TECHNICAL BACKGROUND

The technical background of the present invention is that of systems and methods for monitoring the progress of a pregnancy or childbirth of a pregnant woman, in order to check whether the fetus is following a normal or irregular course. More particularly, the invention relates to a system and method for assessing a situation that could lead to fetal hypoxia.

Assessment of fetal well-being during pregnancy is largely based on analysis of cardiotocographic data, that is to say fetal heart rate and intensity of uterine contractions. Practitioners—midwives and obstetricians—perform this analysis visually and empirically, following the recommendations of medical associations. This assessment is of the utmost clinical importance, as it makes it possible to detect possible fetal anomalies, and in particular the fetal hypoxia anomaly, and to adapt the mode of childbirth, if necessary, by choosing to maintain a vaginal delivery or to induce an emergency cesarean delivery if the risk of fetal hypoxia is proven, in order to avoid any permanent damage to the fetus.

In the current state of the art, assessment of the risk of fetal hypoxia is based largely on visual analysis of cardiotocographic data, carried out by practitioners in accordance with recommendations from medical associations. These methods are known to be subjective and, ultimately, highly variable, both between different practitioners and between cases studied by the same practitioner. In other words, this method does not provide a sufficiently repeatable and reproducible measurement of the risk of fetal hypoxia.

Surprisingly, despite its great clinical importance, this methodology has not been the subject of any major innovation in recent years, and training tools for cardiotocographic data analysis are non-existent.

However, software programs are known that help automate cardiotocography analysis, some of these software programs being integrated into cardiotocographic measurement devices. In particular, such software can be used to set up real-time alerts based on the recommendations of medical associations. However, such software programs do not clearly measure a risk of fetal hypoxia, making them little used. Generally speaking, these software programs are relatively basic and do not take advantage of recent advances in machine learning.

Also known is the document DOI: 10.1109/ACCESS.2019.2933368 that exploits the particularities of artificial intelligence to analyze cardiotocographic data. Nevertheless, this work requires the use of a very large volume of cardiotocographic data with known fetal outcomes and does not make it possible to determine the reasons and/or justify a potential fetal hypoxia situation that artificial intelligence would predict.

Also known is document CN 114 159 039 A, which describes a method for prenatal fetal heart rate monitoring that comprises a step of preprocessing a fetal heart rate signal, a uterine contraction signal, the age of a pregnant woman, and information relating to the week of gestation, and a step of designing a convolutional neural network to extract reference values from the preprocessed signals.

The disadvantage of the method described in CN 114 159 039 A is that it incorporates an artificial intelligence model for supervised training. It is therefore limited by the number of annotated data, which are not in significant quantity for the prediction of fetal hypoxia.

The artificial intelligence model presented is based on a “conventional” machine learning method known as Light Gradient Boosting Machine. As the annotated input data are limited, the use of this method leads to low-quality interpretation and analysis errors, resulting in a model that is not very advanced and ultimately lacks precision, making it unusable by medical staff.

### INVENTION

The object of the present invention is to propose a new system for assessing a situation that could lead to fetal hypoxia, in order to address the above problems and also lead to other advantages.

Another aim of the invention is to better assess the risk of fetal hypoxia, with a high degree of accuracy, by taking into account all the data available to practitioners for carrying out such an assessment.

Another aim of the invention is to provide a set of metrics to support obstetric examinations, enabling medical staff to identify the cause of computed fetal hypoxia in variations in fetal heart rate and the uterine contraction signal.

Another aim of the invention is to take the clinical context into account when assessing the risk of fetal hypoxia.

Another aim of the invention is to enable practitioners to understand the origin of the assessed risk of fetal hypoxia by providing contributions from multiple parameters to this risk.

Another general aim of the invention is to reduce neonatal morbidity.

According to a first aspect of the invention, at least one of the aforementioned objectives is achieved with a system for evaluating a situation of a given pregnant woman that may lead to fetal hypoxia, the system comprising:
a uterine contraction sensor configured to measure an intensity of uterine contractions in the given pregnant woman;
a heart rate sensor configured to measure a fetal heart rate of at least one fetus carried by the given pregnant woman, the measured fetal heart rate and intensity of uterine contractions forming the cardiotocographic data;
a storage unit for a trained artificial intelligence model using a neural network, connected to a computing and display unit itself connected to the sensors, the artificial intelligence model being trained from at least:
an annotated “with fetal outcome” database comprising pre-processed training cardiotocographic data obtained from pregnant women in labor, and fetal outcome data associated with the training cardiotocographic data, enabling assessment of fetal hypoxia at birth,
an unannotated database, consisting of cardiotocographic data, fetal outcome thereof not necessarily known, especially if recorded more than 1 hour before birth;

Advantageously, the unannotated data can come from: records during labor, regardless of the number of hours before delivery, and/or records from pregnancy monitoring;

Advantageously, the artificial intelligence model can be trained with a maternal and/or fetal clinical training database;

The computing and display unit being configured to enable, subsequently to the training of the artificial intelligence model:
pre-processing cardiotocographic data of the given pregnant woman;
computing a risk of fetal hypoxia at multiple points in time from the cardiotocographic data processed and measured at multiple points in time, and advantageously from the clinical data of the given pregnant woman, on the basis of the trained artificial intelligence model, advantageously owing to the maternal and/or fetal clinical data of the given pregnant woman being entered or recorded beforehand, in order to take these clinical data into account by the computing unit.

In the context of the present invention, the system according to the first aspect of the invention is suitable for use in particular in the following situations:
for use in the delivery room, during the labor and childbirth of the pregnant woman, to help practitioners determine in real time the risk of fetal hypoxia, and define the best course of action for the pregnant woman and her fetus;
for use in pregnancy monitoring, particularly in high-risk pregnancies, in the weeks leading up to childbirth, that is to say when the pregnant woman is not in labor. When used in this way, the system according to the first aspect of the invention makes it possible to verify that the cardiotocographic data and the clinical data are reflecting good fetal development;
for use in training practitioners in the context of childbirth simulation, to enable them to sharpen their observations and interpretation of cardiotocographic data for assessing the risk of fetal hypoxia, by enabling comparison with a reference magnitude given, for the same situation, by the system according to the first aspect of the invention.

In the context of the invention, the system according to the first aspect comprises one or two measurement and analysis channels, enabling a risk of fetal hypoxia to be determined for a single fetus or for two fetuses, respectively. In the case where the system according to the first aspect of the invention comprises two channels, a first channel is used to measure cardiotocographic data relating to the first fetus, and a second channel is used to measure cardiotocographic data relating to the second fetus. Of course, the clinical data relating to the pregnant woman is common and identical to both assessments carried out by the system according to the first aspect of the invention. Thus, the system according to the first aspect of the invention can, via the first cardiotocographic data and advantageously the clinical data, determine a first risk of fetal hypoxia for the first fetus, and it can also determine a second risk of fetal hypoxia relating to the second fetus via the second cardiotocographic data and advantageously the clinical data.

In the context of the present invention, the storage unit comprises one or more memories, as are present in a computer, calculator, or server for example, and the computing and display unit comprises one or more processors or one or more microcontrollers, and a display device such as a screen for example. Of course, the display device, the computing unit, and the storage unit are all connected to each other, by wired or wireless means, to enable data transfer and display of sensor measurements, cardiotocographic data, and clinical data. The display device, computing unit, and storage unit can be centralized in a single unit, such as a computer or monitor, or they can be distributed—such as on a remote server—and linked together by communication means.

In the context of the present invention, the display device comprises, for example, a communication interface configured to enable a user to enter or record clinical data. The communication interface can take the form of a touch screen, a keyboard, or any other means. The communication interface is of course connected to the computing unit and the storage unit.

In the context of the present invention, the artificial intelligence model is a mathematical model that makes it possible to compute output data representative of the risk of fetal hypoxia, based on input data comprising cardiotocographic data and, in one possible embodiment of the invention, clinical data. To do this, the artificial intelligence model has been trained to converge towards a risk of fetal hypoxia that is consistent with, that is, analogous to, the risk of fetal hypoxia as determined from the metrics used by practitioners to qualify the risk of fetal hypoxia, such as pH measurement, preferentially. Obviously, the purpose of the invention is to enable better interpretation than that carried out by practitioners, with earlier, more reliable, more accurate detection of the risk of fetal hypoxia.

To this end, the mathematical model was trained using annotated cardiotocographic training data for which the state of the fetus delivered by the pregnant woman is qualified by a metric. In other words, the annotated cardiotocographic training data obtained during the pregnant woman's labor and delivery were qualified by the condition of the fetus once born, in order to enable the mathematical model to determine which situations of the cardiotocographic data lead to a healthy delivery of the fetus, and which may sometimes lead to a fetus with sequelae resulting from fetal hypoxia during pregnancy or delivery, or even fetal morbidity and mortality. By way of a non-limiting example, the qualification of the annotated cardiotocographic training data can take the form of the formation of a pair of values comprising, on the one hand, the cardiotocographic data and, on the other hand, a value reflecting the fetal hypoxia of the delivered fetus, such as, for example, a pH measurement. In addition, this data qualification via fetal outcome is also supplemented by maternal and/or fetal clinical data, to form annotated data sets.

In the context of the invention, maternal clinical data are physiognomic and/or contextual parameters relating to the pregnant woman carrying the fetus being monitored, and fetal clinical data are physiognomic and/or contextual parameters relating to the fetus.

In the context of the present invention, the system according to its first aspect enables, successively,
pre-processing the cardiotocographic data measured on the pregnant woman, forming the processed cardiotocographic data;
in one possible embodiment of the invention, capturing and recording the pregnant woman's clinical data;
computing the risk of fetal hypoxia from the processed cardiotocographic data and, in one possible embodiment of the invention, from the clinical data using the trained artificial intelligence model, that is, the model that had been trained using the annotated cardiotocographic training data, as described above.

In the context of the invention, the risk of fetal hypoxia assessed by the system includes quantification of a risk level associated with the identification of parameters representative of fetal hypoxia.

The system of the invention advantageously comprises at least one of the following improvements, the technical features forming these improvements being able to be taken alone or in combination:
the maternal and/or fetal clinical training data for the given woman are selected from the following list: maternal data relating to pregnant women, including in particular an age; and/or fetal data relating to the fetus, including in particular: a gestational age, an estimate of a fetal weight, such as for example the determination of a quantile of the fetal weight in relation to a distribution of fetal weight as generally observed, for this given term of pregnancy, in a given reference population. More generally, the maternal data relating to the pregnant woman may comprise age, body mass index, parity, indication of a singleton or twin pregnancy; and/or the fetal data relating to the fetus may comprise gestational age, sex, estimated fetal weight; and/or delivery data such as, for example, a suspected intrauterine infection, obtained for example by measuring the pregnant woman's temperature, the presence of gestational diabetes or the presence of a vascular pathology, such as, for example, gestational hypertension, pre-eclampsia, or a cervical dilatation measurement;
to enable optimal processing of the measured data, the computing and display unit pre-processes the cardiotocographic data. In particular, pre-processing involves filling in missing cardiotocographic data and/or deleting outlier cardiotocographic data. In particular, cardiotocographic data completion involves, for example, linear interpolation between cardiotocographic data directly preceding the missing cardiotocographic data and cardiotocographic data directly succeeding the missing cardiotocographic data. Additionally, the removal of outlier cardiotocographic data comprises an analysis of a deviation between the outlier cardiotocographic data and the cardiotocographic data directly adjacent to the outlier cardiotocographic data, the outlier cardiotocographic data being removed if the deviation is greater than a threshold value;
the artificial intelligence model is trained using pre-processed cardiotocographic training data. In addition, the artificial intelligence model can also be trained using indicators computed from pre-processed cardiotocographic data, characterizing the course of the heart rates of the fetus. These indicators are determined so as to reproduce the analysis made by practitioners of the fetal situation, in accordance with the recommendations of medical associations. These indicators are computed on the basis of cardiotocographic data measured on pregnant women, in order to qualify certain visual and graphic characteristics of fetal heart rates and/or uterine contractions, in accordance with the know-how and examination habits recommended by medical associations;
the indicators are preferentially selected from the following list: (i) a baseline, computed as a sliding average of the fetal heart rate, excluding periods of acceleration and deceleration, and/or (ii) a duration and number of critical cardiac events of the acceleration type, each critical cardiac event of the acceleration type being determined by a fetal heart rate above a nominal heart rate range defined around the baseline, and/or (iii) a duration and number of critical cardiac events of the deceleration type, each critical cardiac event of the deceleration type being determined by a fetal heart rate below the nominal heart rate range defined around the baseline, and/or (iv) a variability of the fetal heart rate. These indicators are those recommended by medical associations for detecting and/or qualifying a risk of fetal hypoxia;
the variability of the fetal heart rate is determined for a plurality of different time scales. In particular, in the context of the invention, the variability of the fetal heart rate is defined by a standard deviation of said heart rate or by the derivative of the fetal heart rate. In addition, we define (i) short-term heart rate variability, that is, over a period of a few seconds, for example between 5 seconds and 60 seconds, and (ii) long-term heart rate variability, that is, of the order of a few minutes, for example between 5 minutes and 25 minutes;
additionally, the indicators may also comprise a relative position of the baseline with respect to an expected baseline reference. In particular, the indicators may comprise the identification of a baseline position that is too low with respect to the expected reference of said baseline, and/or a baseline position that is too high with respect to the expected reference of said baseline, and/or a number of critical cardiac events of the fetal heart rate deceleration type lasting less than 2 minutes, and/or a number of critical cardiac events of the fetal heart rate deceleration type lasting more than 2 minutes, and/or a number of critical cardiac events of the fetal heart rate acceleration type;
in the context of the invention, the neural network is a model for predicting a variable of interest—in this case, the risk of fetal hypoxia—from explanatory variables—in this case, cardiotocographic data and, where applicable, clinical data. The neural network is optimized on a retrospective database. In particular, the neural network implemented by the system is of the convolutional or transform type based on the attention mechanism, as it is particularly suited to processing temporal signals, including cardiotocographic data;
in the context of the invention, and more particularly in the context of using the system according to the invention for training purposes, the artificial intelligence model comprises a generative model which makes it possible to generate fictitious data similar to that available on the databases on which the neural network has been trained, making it possible here to generate signals representative of annotated cardiotocographic data. The artificial intelligence model is a neural network that has been trained in semi-supervised training from (i) a first non-annotated database containing only the first pre-processed training cardiotocographic data obtained for pregnant women, with no data on the fetal outcome associated with these training cardiotocographic data and, then (ii) a second annotated database “with fetal outcome” comprising second pre-processed cardiotocographic training data obtained for pregnant women, and fetal outcome data enabling assessment of fetal hypoxia at birth associated with these cardiotocographic training data, and in one possible embodiment (iii) maternal and/or fetal clinical training data. Thus, the artificial intelligence model is trained with cardiotocographic data for which the fetal outcome is unknown—the data from the first database—and annotated data resulting from delivery situations for which the fetal outcome has been determined—resulting from the second database. The data from the first database may have been obtained during obstetrical visits prior to delivery, or during early stages of labor far removed from the birth itself, e.g. prior to 1 hour before the delivery, which enables the artificial neural network to be trained with a very large quantity of cardiotocographic data (the neural network is, as it were, pre-trained by these thousands of unannotated data to all possible evolutions of the cardiotocographic data), so that in a second stage, this neural network is then better able to be trained more quickly and more efficiently with the data from the second annotated database;
these semi-supervised training methods are used because there is access to both a limited amount of annotated data—cardiotocographic data for which the fetal outcome is known and associated with said cardiotocographic data—and a larger amount of unannotated data—cardiotocographic data for which the fetal outcome is unknown;
the neural network is first trained with the first unannotated database to obtain processing blocks of pre-processed cardiotocographic data (using convolutional or transform-based architecture), based solely on the cardiotocographic data. In a second step, the neural network is trained with data from the second annotated database, the “with fetal outcome” database, by initializing the pre-processed cardiotocographic data processing blocks as learned in the first step. In the context of the invention, blocks are intermediate states of computation implemented by the artificial intelligence model and, more specifically, by the neural network. In other words, they are neurons located between an input and an output of the neural network;
the computing and display unit advantageously comprises the neural network;
in particular, the neural network is configured to (i) apply a transformation, e.g. convolutional, of the processed cardiotocographic data, forming experimental data, e.g. convolved data, (ii) apply a concatenation to these experimental data with the clinical data of the pregnant woman, together forming concatenated data, and (iii) apply a prediction of the risk of fetal hypoxia from the concatenated data;
the computing and display unit is configured to, at a given display frequency, (i) compute and display a series of indicators characterizing the fetal heart rate of the fetus of the given pregnant woman, interpretable by practitioners, these indicators being displayed simultaneously with the fetal hypoxia risk computed by artificial intelligence of the given pregnant woman and/or (ii) compute, by application of a mathematical regression model, a statistical fetal risk, and (iii) compute a contribution of these indicators to the statistical fetal hypoxia risk and simultaneously display the fetal hypoxia risk computed by artificial intelligence and a contribution of these indicators. It is understood here that, in the context of the invention, the indicators characterizing the fetal heart rate are of the type mentioned above and as defined by medical associations. In other words, the indicators computed here are of the type that can be interpreted by practitioners. In the context of the present invention, the operations carried out by the computing and display unit are performed according to at least one predetermined sampling frequency and/or display frequency. The sampling frequency—used to determine or measure the fetal heart rate and the intensity of uterine contractions—may comprise a first sampling frequency used to determine or measure the fetal heart rate and a second sampling frequency used to determine or measure the intensity of uterine contractions. Advantageously, the computing and display unit uses a single sampling frequency, generally of the order of a few hertz, for example 4 Hz. In addition, the computing and display unit finally computes one or more indicators of the risk of fetal hypoxia, as well as their contribution to said risk, depending on the display frequency. As a non-limiting example, the display frequency is of the order of a few Hertz, for example 1 Hz;
the computing and display unit is configured to display, at the display frequency, the contribution of these indicators to the fetal hypoxia risk of the given pregnant woman, computed by the trained artificial intelligence model. Additionally or alternatively, the computing and display unit is configured to display, at the display frequency, the fetal hypoxia risk of the given pregnant woman, computed by the trained artificial intelligence model;
in the context of the invention, the mathematical regression model is of the type that makes it possible to determine a weighting coefficient for each parameter involved in computing the indicators and/or the risk of fetal hypoxia. In particular, the mathematical regression model is, for example, of the logistic regression type;
the artificial intelligence model is trained on at least one of the following data representative of fetal outcome: (i) a pH measurement on a fetal umbilical cord, with a pH value below 7.15 qualifying moderate or severe fetal hypoxia, and a pH value below 7.05 qualifying severe fetal hypoxia; and/or (ii) gasometry measurements on the umbilical cord, for example to demonstrate a base concentration greater than 12 mmol/L or a lactate concentration greater than 6 mmol/L; and/or (iii) an Apgar score; and/or (iv) possible hospitalization of the fetus in intensive care for suspected fetal hypoxia;
the artificial intelligence model is trained on cardiotocography recordings of pregnant women comprising (i) a first, non-annotated database, comprising recordings located before the delivery of the pregnant women, (ii) a second, annotated database called “with fetal outcome”, comprising recordings located at most 40 minutes, advantageously at most 30 minutes, from the birth of the fetus carried by these pregnant women and annotated with fetal outcome data, and (iii) maternal and/or fetal clinical data for these women for whom the fetal outcome of their delivery is known. The first unannotated database comprises data augmented by time-shifting;
the system comprises communication means between, on the one hand, the uterine contraction sensor and the heart rate sensor and, on the other hand, the computing and display unit, so that the system can be used on an outpatient basis or at home, the computing and/or display of the risk of fetal hypoxia being designed to be monitored remotely by a practitioner.

According to a second aspect of the invention, a method is proposed for assessing a risk of fetal hypoxia of a fetus of a given pregnant woman, using a system according to the first aspect of the invention or according to any of its improvements, the method comprising the following steps:
a step for acquiring cardiotocographic data and maternal and/or fetal clinical context data from the given pregnant woman;
a step of pre-processing the cardiotocographic data of the given pregnant woman;
a step for computing the given pregnant woman's risk of fetal hypoxia by applying a trained artificial intelligence model.

The method according to the second aspect of the invention advantageously comprises at least one of the following improvements, the technical features forming these improvements being able to be taken alone or in combination:
the preprocessing step comprises (i) a step for filling in missing cardiotocographic data; and/or (ii) a step for deleting outlier cardiotocographic data;
the maternal or fetal clinical data are selected from the following list: (i) maternal data relating to pregnant women, including in particular age, body mass index, parity, indication of a singleton or twin pregnancy, and/or (ii) fetal data relating to fetuses, including in particular gestational age, estimated fetal weight, sex, and/or delivery data such as, in particular a suspected intrauterine infection, qualified for example by measurement of a temperature of the pregnant woman, the presence of a vascular pathology such as, for example, gestational hypertension, pre-eclampsia, the presence of gestational diabetes, a cervical dilatation measurement;
the method comprises a step of computing and displaying: (i) indicators interpretable by practitioners characterizing the fetal heart rate of the fetus of the given pregnant woman, a statistical fetal risk by applying a mathematical regression model, and simultaneously a contribution of these indicators to the risk of fetal hypoxia of the given pregnant woman computed by the trained artificial intelligence model.

Various embodiments of the invention are envisaged, incorporating in all their possible combinations the various optional features set out herein.

Further features and advantages of the invention will become apparent from the following description, on the one hand, and from a number of illustrative and non-limiting embodiments with reference to the attached schematic drawings, on the other hand, in which:

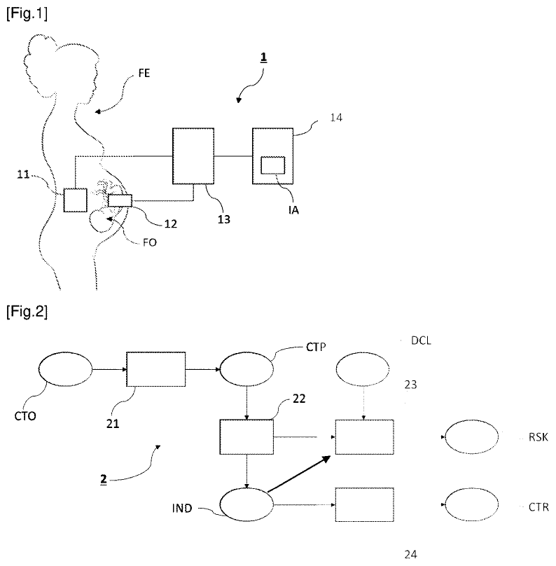

FIG. 1 shows a schematic view of the system according to the first aspect of the invention;

FIG. 2 shows a synoptic view of the operation of the system according to the invention;

FIG. 3 shows a comparison of the performance of the system according to the invention with diagnoses made by practitioners;

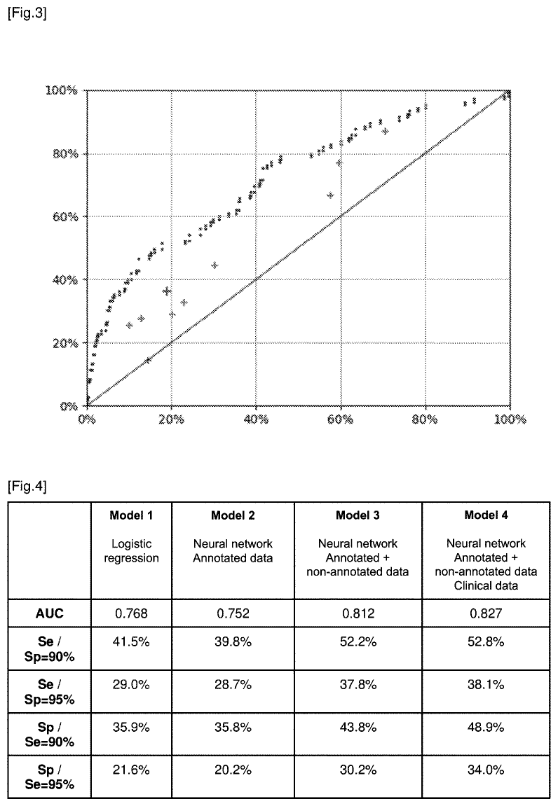

FIG. 4 shows a comparison of the performance of the system according to the invention in different embodiments;

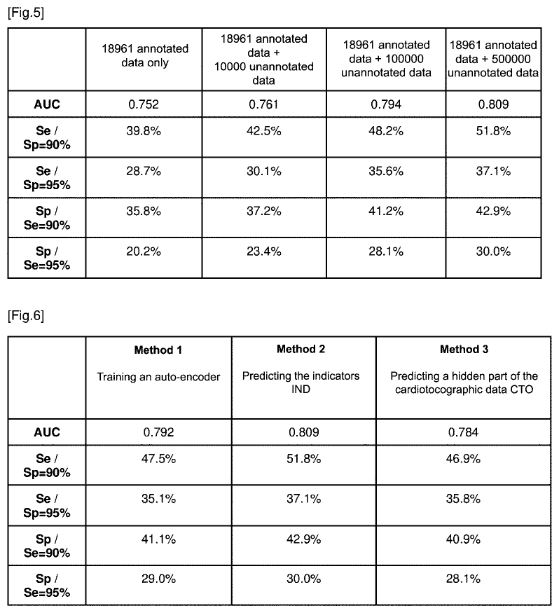

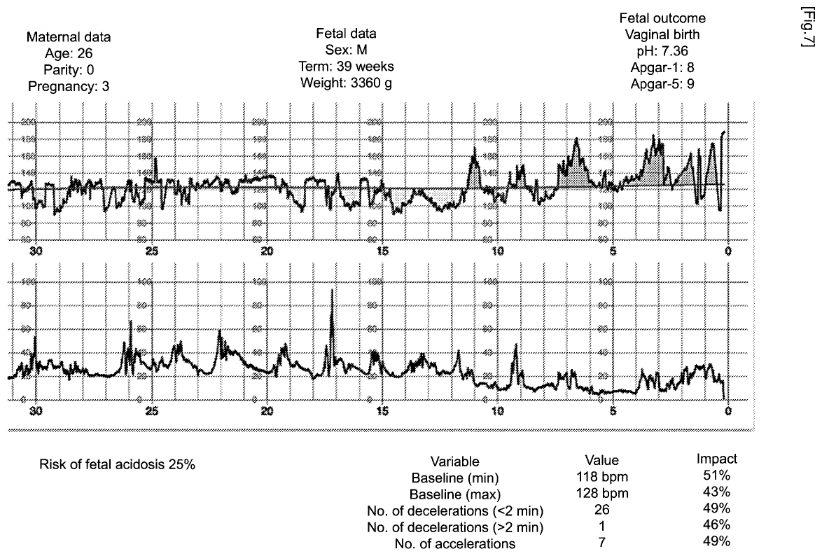

FIG. 5 shows a comparison of the performance of the system according to the invention, using a variable number of unannotated data with a constant number of annotated data; a cardiotocographic data corresponds, by way of illustration only and without limitation, to a signal sequence, for example of 30-minute duration, for a pregnant woman, this cardiotocographic data being labeled by the fetal outcome (second database) or not (first database);

FIG. 6 shows the differences in results obtained with the different semi-supervised training methods, using the same amount of cardiotocographic data;

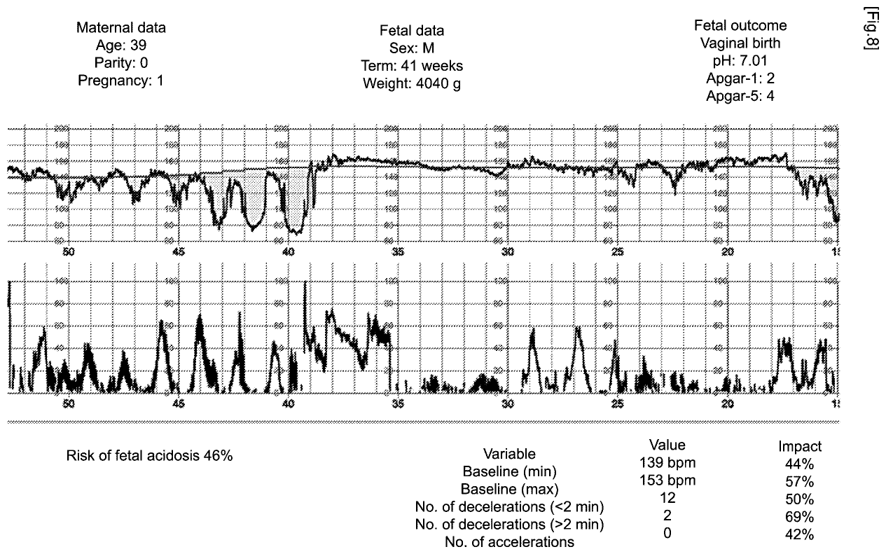

FIG. 7 shows a first visual representation of the data displayed by the system according to the invention and the determination of a first risk of fetal hypoxia;

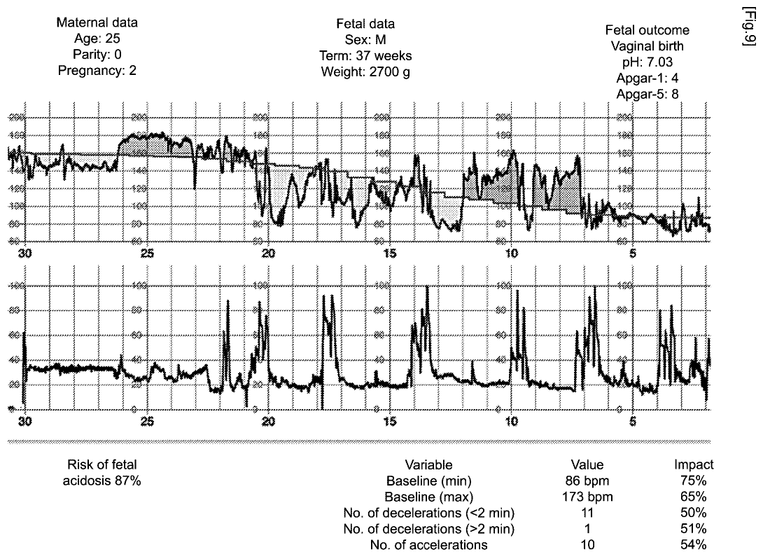

FIG. 8 shows a second visual representation of the data displayed by the system according to the invention and the determination of a second risk of fetal hypoxia;

FIG. 9 shows a third visual representation of the data displayed by the system according to the invention and the determination of a third risk of fetal hypoxia.

Of course, the features, variants, and different embodiments of the invention can be combined with one another in various ways, as long as they are not incompatible or mutually exclusive. In particular, it is possible to imagine variants of the invention comprising only a selection of features described hereinafter in isolation from the other features described, if this selection of features is sufficient to confer a technical advantage or to differentiate the invention from the prior art.

In particular, all the variants and embodiments described can be combined with each other, provided there are no technical obstacles to such combination.

In the FIGURES, elements common to multiple FIGURES retain the same reference.

Assessment of fetal well-being during pregnancy is based on analysis of cardiotocographic data CTO, including measurement of fetal heart rate and intensity of uterine contractions.

At present, this interpretation is made on an individual, empirical and visual basis by practitioners following the recommendations of medical associations, during obstetric examinations to monitor the progress of the pregnancy or to direct the childbirth of a pregnant woman FE. These interpretations of cardiotocographic data CTO have proven to be of the utmost clinical importance, as they make it possible to detect the main fetal anomalies, and in particular fetal hypoxia. These practitioners can then adapt the mode of delivery—vaginal or cesarean—to avoid permanent damage to the fetus FO.

The system 1 as schematically shown in FIG. 1 enables a method 2—visible in FIG. 2—to be implemented for assessing a risk of fetal hypoxia RSK of a pregnant woman FE by automatically analyzing cardiotocographic data CTO and clinical data DCL of the pregnant woman FE. The system 1 thus provides practitioners with an assessment of the risk of fetal hypoxia RSK—measured between 0% and 100%—and highlights the contributions CTR of several indicators IND recognized by medical associations in the measurement of this risk, each weighting being between 0% and 100% and their sum adding up to 100%.

In particular, as can be seen in FIG. 2, the risk of fetal hypoxia RSK is assessed by an artificial intelligence model IA implemented by a neural network 23 of the computing and display unit 13. In addition, the system 1 according to the invention implements a second mathematical model, typically of the logistic regression type 24, in order to evaluate the contribution to the risk of fetal hypoxia RSK of the various indicators IND computed during a calculation step 22 and taken from the recommendations of medical associations and computed automatically on the basis of cardiotocographic data CTO and possibly clinical data DCL.

Particularly advantageously, these models are learned from a retrospective database comprising annotated fetal data for a large number of deliveries, stored in a first database, enabling assessment of fetal hypoxia situations in relation to cardiotocographic data CTO obtained just before delivery. These training data were obtained from preliminary measurements and analyses carried out by the practitioners beforehand. They enable subsequent use of the system 1 and the artificial intelligence model IA in novel situations where the artificial intelligence model IA is thereafter able to correctly predict situations, indicators IND, and the risk of fetal hypoxia RSK.

The system 1, via its computing and display unit 13, updates in real time and at a given display frequency the determined fetal hypoxia risk RSK and the contributions CTR identified in the computation step 22 for the various indicators IND, to reflect the latest available cardiotocographic data and provide practitioners with a decision-support tool.

In the context of the present invention, clinical or medical status is not considered. The system 1 and the method 2 according to the invention simply process technical signals resulting from and/or computed from sensors 11, 12 or instruments.

More particularly, the system 1 according to the first aspect of the invention comprises:
a uterine contraction sensor 11 configured to measure an intensity of uterine contractions in the given pregnant woman FE;
a heart rate sensor 12 configured to measure a fetal heart rate of at least one fetus FO carried by the given pregnant woman FE, the measured fetal heart rate and intensity of uterine contractions forming the cardiotocographic data CTO;
a storage unit 14 for a trained artificial intelligence model IA using a neural network, connected to a computing and display unit 13 itself connected to the sensors 11, 12.

The computing and display unit 13 is configured to implement the artificial intelligence model IA, the computing and display unit 13 particularly comprising a neural network 23.

In the context of the invention, the artificial intelligence model IA is trained on the basis of a first, non-annotated database and a second, annotated “fetal outcome” database, comprising pre-processed training cardiotocographic data CTO obtained from pregnant women in labor, and fetal outcome data associated with the training cardiotocographic data CTO, enabling assessment of fetal hypoxia at birth;
and in one possible variant of the invention, additionally by maternal and/or fetal training clinical data DCL.

Subsequently, the computing and display unit 13 of the system 1 according to the invention is configured to allow:
pre-processing of the cardiotocographic data CTO of the given pregnant woman FE, forming the pre-processed cardiotocographic data CTP;
in the case of the possible variant involving the entering or recording of clinical data DCL of the given pregnant woman FE, the clinical data DCL being taken into account by the computing unit;
the computing of a risk of fetal hypoxia RSK from processed cardiotocographic data CTO, and clinical data DCL of the given pregnant woman FE, using the trained artificial intelligence model IA.

In particular, pre-processing involves filling in missing cardiotocographic data CTO, for example by linear interpolation between cardiotocographic data directly preceding the missing cardiotocographic data and cardiotocographic data directly succeeding the missing cardiotocographic data. In other words, cardiotocographic data are missing if there is a “gap” between two or more adjacent cardiotocographic data CTO. In the context of the invention, such a gap of cardiotocographic data CTO of duration below a threshold value, typically equal to 5 minutes for example, is filled and supplemented, preferentially by linear interpolation. On the other hand, a gap in cardiotocographic data CTO lasting longer than the threshold value is not supplemented.

Alternatively or additionally, the pre-processing comprises a suppression of outlier cardiotocographic data CTO, for example by analyzing a deviation between the outlier cardiotocographic data and the cardiotocographic data CTO directly adjacent to the outlier cardiotocographic data, the outlier cardiotocographic data being suppressed if the deviation is greater than a threshold value. A cardiotocographic value x(t) representative of a state at a given instant is considered an outlier if the absolute value of the quantities x(t)−x(t−1) and x(t)−x(t+1) are, together, greater than a threshold, chosen, for example, as being equal to 30 bpm for fetal heart rate and 20 mm Hg for uterine contractions, where x(t−1) and x(t+1) are respectively the cardiotocographic data CTO preceding and succeeding the given cardiotocographic value x(t). The detected outlier values are then replaced by linear interpolation.

The clinical data DCL of the pregnant woman FE help define the clinical context of the delivery and/or of the pregnant woman FE. The clinical data DCL taken into account by the system 1 according to the invention comprise in particular the following data:
maternal data, such as age, body mass index, parity, and whether the pregnancy is a singleton or twin pregnancy;
fetal data, such as gestational age, sex, estimated fetal weight, etc;
delivery data, such as suspected intrauterine infection, vascular pathology such as gestational hypertension, pre-eclampsia, gestational diabetes, a cervical dilatation measurement.

The system 1 according to the invention make it possible to implement:
cardiotocographic data CTO and clinical data DCL used as input to the system 1;
the computing of indicators IND interpretable by practitioners using a logistic regression model 24 based on the recommendations of medical associations, the indicators IND being computed on the basis of cardiotocographic data CTO and/or clinical data DCL obtained;
the computing of the risk of fetal hypoxia RSK and the computing 22 of the contributions CTR made by each previously computed indicator IND to said fetal hypoxia risk.

Generally speaking, the contributions of indicators IND that can be interpreted by practitioners are computed by the logistic regression model 24, which implements the recommendations of medical associations, on the basis of real-time analysis of cardiotocographic data CTO, including fetal heart rate. The indicators IND computed are as follows:
a baseline, computed as a sliding average of the fetal heart rate, excluding periods of acceleration and deceleration, and/or
a duration and number of critical cardiac events of the acceleration type, each critical cardiac event of the acceleration type being determined by a fetal heart rate above a nominal heart rate range defined around the baseline. In particular, the indicators IND may comprise the identification of a baseline position that is too high relative to an expected baseline reference, such as a number of critical cardiac events of the fetal heart rate acceleration type;
a duration and number of critical cardiac events of the deceleration type, each critical cardiac event of the deceleration type being determined by a fetal heart rate below the nominal heart rate range defined around the baseline. In particular, the indicators IND may comprise the identification of a baseline position that is too low with respect to the expected reference of said baseline, such as for example a number of critical cardiac events of the fetal heart rate deceleration type lasting less than 2 minutes, and/or a number of critical cardiac events of the fetal heart rate deceleration type lasting more than 2 minutes; and/or
short-term heart rate variability, that is, over a period of a few seconds, for example between 5 seconds and 60 seconds and/or
long-term heart rate variability, that is, on the order of a few minutes, for example between 5 minutes and 25 minutes; and/or

These indicators IND are computed at a given display frequency, enabling the practitioner to monitor them in real time, with values that are always up-to-date.

In the context of the invention, the risk of fetal hypoxia RSK is computed by the artificial intelligence model IA and implemented by the neural network 23. The neural network 23 takes as input variables:
the processed cardiotocographic data CTO, that is, after removal of missing values and filtering of outlier values;
potentially the clinical data DCL if available;
where applicable, the indicators IND computed by the mathematical logistic regression model 24 and based on the recommendations of medical associations, as described above. More specifically, the mathematical logistic regression model 24 makes it possible to determine the contribution of each indicator IND computed in the prediction of the risk of fetal hypoxia RSK.

In the context of the invention, and as previously mentioned, the artificial intelligence model IA is implemented by a neural network 23 of the computing and display unit 13. The neural network 23 is more particularly of the type of a convolutional network or a transformer based on the attention mechanism or a recurrent network.

The transformer is described, for example, in the publication described at this hyperlink https://arxiv.org/abs/1706.03762.

The “recurrent networks are in particular of the LSTM type” described at this hyperlink: https://www.researchgate.net/publication/13853244_Long_Short-term_Memory)

As previously mentioned, the neural network 23 is configured to determine a magnitude representative of a risk of fetal hypoxia RSK. This representative variable is a positive real number between 0, synonymous with a total absence of risk of fetal hypoxia RSK, and 1, associated with a definite prediction of fetal hypoxia. In other words, the neural network 23 is configured to determine a prediction of risk of fetal hypoxia RSK from the following data:
the cardiotocographic data CTO, which are integrated into the neural network 23 in a first step. As previously mentioned, this cardiotocographic data CTO is determined by the associated sensors 11, 12 and at a sampling frequency of the order of a few hertz. By way of a non-limiting example, the measurements provided by the fetal heart rate sensor 12 and by the uterine contraction sensor 11 are taken over a given period whose length depends on the context in which the system 1 according to the invention is implemented. By way of a non-limiting example, in the case of use during childbirth, the measurement time may be of the order of several hours, preferentially from the start of the labor of the pregnant woman FE, and even more preferentially of the order of several tens of minutes—typically between 20 min and 30 min before the end of delivery, that is, the birth of the fetus FO;
clinical data DCL, which are integrated into the neural network 23 in a second step and reflect the clinical context associated with the pregnant woman FE and/or the fetus FO and/or the pregnancy, as described above.

In a third step, the artificial intelligence model IA applies processing blocks, for example convolutional ones, to the cardiotocographic data CTO in order to transform said cardiotocographic data CTO received in the first step in the form of a matrix of dimensions (N,2), where N is a natural integer, into a vector, for example a convolved one, of dimension M, where M is a natural integer less than N with advantageously N/M being greater than or equal to 10.

In a fourth step, the artificial intelligence model IA summarizes the clinical context of the pregnant woman FE and/or fetus FO and/or pregnancy in a vector representative of the clinical data DCL.

In a fifth step, the artificial intelligence model IA then concatenates the dimension vector M computed in the third step with the vector representative of the clinical data DCL determined in the fourth step.

In a sixth step, the artificial intelligence model IA operates the concatenated vector determined during the fifth step in dense layers of the neural network 23, in order to ultimately determine a prediction of the risk of fetal hypoxia RSK.

Of course, in the context of the present invention, the expected performance of the system 1 according to the first aspect of the invention depends on the ability of the artificial intelligence model IA to predict a risk of fetal hypoxia RSK that at least converges with the diagnoses made by practitioners, and preferentially converges with the meaning of the metrics used to qualify the risk of fetal hypoxia, such as preferentially a pH measurement. Of course, these performances depend on the developed artificial intelligence model and its training process, which will now be described.

Generally speaking, the training of the neural network 23 corresponds to the optimization of its internal parameters using a retrospective database containing, for a large number of childbirths—that is, in excess of several thousands or even several tens of thousands—the following information:
cardiotocographic data CTO prior to childbirth;
clinical data DCL reflecting the maternal-fetal clinical context;
information qualifying the fetal outcome in order to assess at least the presence or absence of fetal hypoxia.

In the context of the present invention, the envisaged training method of the neural network 23 is based on semi-supervised training, enabling advantage to be taken of more and more diverse, unannotated cardiotocographic data CTO.

A supervised method used in the prior art cannot exploit all available cardiotocographic data CTO, since many cardiotocographic data CTO exist without being associated with a qualified fetal outcome:
for example because it was not filled out at the time of childbirth;
for example because the cardiotocographic data CTO were measured during a follow-up obstetric examination during the pregnancy of the pregnant woman FE;
for example, because the cardiotocographic data CTO were measured during childbirth but well before the last half-hour prior to the birth of the fetus FO.

Also, the training method according to the invention, implementing semi-supervised training, makes more data available for training the neural network 23 and thus leads to a very high accuracy of said neural network 23 thus trained, compared to a supervised training method.

The semi-supervised method according to the invention consists of two successive stages:
an initial step of training the pre-processed cardiotocographic data processing blocks, for example convolutional, of the neural network 23 on all the available data—annotated or not annotated, the initial training step making it possible to determine first values for the parameters qualifying the blocks, for example convolutional, of the neural network 23;
a second step for training the neural network 23, wherein the parameters of the blocks of the neural network 23 are predetermined and equal to the parameters learned in the previous step.

The blocks are trained using one of the following three methods, in order to ultimately build blocks capable of interpreting the cardiotocographic data CTO and summarizing it in a vector of size M. (1) Training an auto-encoder, which takes the form of a model whose objective is to reproduce the data that has been provided as input. (2) Training a model whose aim is to predict indicators IND, or a subset of them, from data provided as input. (3) Training a model, wherein certain parts of the signal are masked, and the aim is to predict them from the rest of the signal.

During the initial training step, knowledge of the fetal outcome associated with the cardiotocographic data CTO is not required, allowing the model to be trained on a larger set of cardiotocographic data CTO.

The initial training of the cardiotocographic data CTO processing blocks of neural network 23 using one of the 3 methods described comprises the following steps:
a step for integrating cardiotocographic data CTO from a training database, possibly partially masked for method 3, into the neural network 23;
a step of applying the blocks, e.g. convolutional blocks, to the cardiotocographic data CTO in order to transform them in the form of a matrix of dimensions (N,2), where N is a natural integer and 2 corresponds to the two different types of cardiotocographic data (uterine contractions, fetal heart rate), into an e.g. convolved vector of dimension M, where M is a natural integer much smaller than N (M<<N);
a step wherein various blocks are applied to construct a prediction from the vector of dimension M: the cardiotocographic data CTO in method 1, a vector containing indicators IND in method 2, or the cardiotocographic data that has been masked in method 3.

Upon completion of the initial training step via one of the 3 methods described, the second training step is implemented in order to use the neural network 23 with new cardiotocographic data CTO of vectors (N, 2) and by configuring said neural network 23 so that the structural parameters of the processing blocks of the pre-processed cardiotocographic data are initialized to the values determined during said initial training step. This makes it possible to obtain one-dimensional column vectors of size M, with M<<N (advantageously N/M greater than or equal to 10), from cardiotocographic data (uterine contractions, fetal heart rate) of dimensions (N, 2) and to possibly concatenate these column vectors (which faithfully describe and summarize the evolution of cardiotocographic data due to the previously trained encoder) with clinical data. During the second training step, the aim is now to optimize the other parameters of the neural network 23 so that the fetal outcome predicted by it is as close as possible to the actual fetal outcome, known for the annotated database.

Thus, according to the invention, the use of the artificial intelligence model IA implemented by the trained neural network 23 enables risk of fetal hypoxia RSK to be predicted accurately.

Additionally, the invention also addresses the determination of indicators IND—computed on the basis of recommendations from medical associations—and enabling certain aspects of an risk of fetal hypoxia RSK to be highlighted and interpreted by practitioners. The aim here is to compute these metrics and make them available to practitioners for subsequent medical interpretation and decision-making.

Thus, the system 1 according to the first aspect of the invention implements a second model, in parallel to the artificial intelligence model IA as previously described, the second model being used to compute indicators IND in real time from cardiotocographic data CTO.

The aim of this second model is, on the one hand, to identify indicators IND that are well-known and widely used by practitioners and, on the other hand, to compute a risk of fetal hypoxia RSK, based on these indicators IND, while determining the share of each indicator IND in the risk of fetal hypoxia RSK thus computed.

In a particularly advantageous way, the model implemented in the context of the present invention is more particularly a logistic regression model 24, wherein the risk of fetal hypoxia RSK P is defined as

$$P = \frac{{exp}^{( {β}_{0} + {\sum }_{i = 1}^{n} {β}_{i} {x}_{i} )}}{1 + {exp}^{( {β}_{0} + {\sum }_{i = 1}^{n} {β}_{i} {x}_{i} )}} .$$

where:
the estimated risk of fetal hypoxia RSK, P, is a real number between 0 and 1, preferentially expressed as a percentage;
x1, . . . , xn correspond to the indicators IND used as model inputs and derived from the recommendations of medical associations;
the β coefficients are estimated on the retrospective database used to train the neural network 23.

As a result, a contribution Ci of each indicator IND to the risk of fetal hypoxia RSK is computed, according to the following formula:

$${C}_{i} = \frac{{β}_{i} {x}_{i}}{{\sum }_{i , {β}_{i} {x}_{i} > 0} {β}_{i} {x}_{i}} .$$

This indicator IND is only computed and displayed for variables for which the product βᵢxᵢ is greater than 0. These variables are thus associated with a positive contribution to the risk of fetal hypoxia RSK. In addition, the sum of all indicator contributions is equal to 1.

As an example, consider a case in which the risk of fetal hypoxia assessed by the artificial intelligence model is rated 70%, the risk assessed by the second model is rated 78%, and the contributions associated with the indicators, computed as detailed above and based on the second statistical model, are positive and rated 31% and 69% respectively for the number of accelerations and the number of decelerations. The system will then display 3 values: the risk from the artificial intelligence model (70%), and the two contributions from the number of accelerations and decelerations (31% and 69%).

Advantageously, the risks estimated by the two models are generally close enough that the contributions computed from the second model can confidently be used with the risk computed by the artificial intelligence model.

With reference to FIG. 3, the performance of the system 1 according to the invention is shown by means of a test performance characteristic curve (Receiver Operating Characteristic, or ROC, curve), in this case that of the risk of fetal hypoxia RSK. More specifically, FIG. 3 shows an evaluation of the performance of the risk of fetal hypoxia RSK measurement predicted by the system 1 according to the invention and in comparison with diagnoses made
in comparable circumstances, that is, on close or analogous sets of cardiotocographic data CTO—by practitioners for the assessment of the same risk of fetal hypoxia RSK.

The graph shown in FIG. 3 represents:
on the y-axis, the true positive rate, that is, the proportion of fetal hypoxia situations correctly detected by the system 1 according to the invention; and
on the x-axis, the false positive rate, that is, the proportion of situations without fetal hypoxia for which the system 1 according to the invention had predicted fetal hypoxia.

In the graph shown in FIG. 3, each point on the curve corresponds to a quantification of the risk of fetal hypoxia RSK determined by the system 1 according to the invention.

As can be seen from the graph shown in FIG. 3, the evaluation of the system 1 according to the invention is carried out via a reference database containing, for 552 clinical cases, cardiotocographic data CTO, the fetal outcome enabling evaluation of a fetal hypoxia situation, defined by the condition (pH<7.15), and the final diagnosis given by obstetricians who indicated, for each case, whether, in their opinion, a situation of fetal hypoxia would arise during childbirth. The evaluation of the annotations given by each obstetrician is represented by a cross.

In FIG. 3, it can be seen that the performance produced by the system 1 according to the first aspect of the invention is perceived as having higher reliability and accuracy of predicted risk of fetal hypoxia RSK than the practitioners' own diagnoses.

FIG. 4 shows the performance of different models on a database containing 18961 cases. Model 1 is a logistic regression using indicators IND, and can be likened to the second model on which the computation of indicator contributions is based. Model 2 is a neural network similar to that of system 1, trained only using annotated data. Model 3 is a neural network trained on both annotated and non-annotated data as described above. More precisely, the model is trained using around 580000 unannotated data (for 18961 annotated cases). Model 4 also includes clinical data. Performance is assessed using several metrics: AUC (Area Under the ROC Curve), sensitivity at fixed specificity (90% or 95%), and specificity at fixed sensitivity (90% or 95%). FIG. 4 shows the significant improvement in model performance by adding the unannotated data (model 3) according to the invention and by adding the clinical data (model 4) according to the invention.

FIG. 5 shows the improvement in model performance, as a function of the number of unannotated data used in training the cardiotocographic data processing blocks, with the same number of annotated data, constant each time. This figure shows the contribution of unannotated data.

FIG. 6 compares the 3 described semi-supervised training methods, using all of the annotated data and 500000 unannotated data, showing the effectiveness of all 3 methods and the superiority of the approach of predicting indicators IND from preprocessed cardiotocographic data.

FIGS. 7, 8 and 9 show multiple real-time measurements and assessments of the risk of fetal hypoxia RSK, as performed by the system 1 according to the invention as described above:
FIG. 7 shows a first situation wherein the risk of fetal hypoxia RSK computed by the artificial intelligence model IA is rather low, equal to 25%, wherein no indicator IND has a positive contribution to the risk of fetal hypoxia;
FIG. 8 shows a second situation wherein the risk of fetal hypoxia RSK computed by the artificial intelligence model IA is moderate, equal to 46%. The number of fetal heart rate decelerations identified on the cardiotocographic data CTO explains 72% of the estimated risk, while the remaining 28% is explained by an abnormally high baseline;
FIG. 9 shows a third situation wherein the risk of fetal hypoxia RSK computed by the artificial intelligence model IA is moderate, equal to 87%. The risk is explained by an abnormally high baseline at times (56%), abnormally low at other times (33%), and to a lesser extent by a high number of accelerations (9%) and prolonged deceleration (2%).

In summary, the invention relates to a system 1 for providing a method for the automated analysis of cardiotocographic data CTO and clinical data DCL to measure a risk of fetal hypoxia RSK, of between 0% for absence of any risk and 100% for confirmed fetal hypoxia. The system 1 according to the invention makes it possible, by means of the artificial intelligence model IA facilitated by the neural network 23 and by means of the mathematical regression model, to determine the contributions CTR to that risk of the various risk indicators IND capable of being interpreted by practitioners. The models for assessing the risk of fetal hypoxia RSK, determined by means of a neural network 23, and the contributions CTR of the various indicators IND used to determine the risk of fetal hypoxia RSK, determined by logistic regression 24, are trained on a retrospective database.

Of course, the invention is not limited to the examples just described, and many adjustments can be made to these examples without departing from the scope of the invention. In particular, the various features, forms, variants, and embodiments of the invention can be combined with one another in various ways, as long as they are not incompatible or mutually exclusive. In particular, all the variants and embodiments described above can be combined with one another.

1. A system for evaluating a situation of a given pregnant woman that may lead to fetal hypoxia, the system comprising:
a uterine contraction sensor configured to measure an intensity of uterine contractions in the given pregnant woman;
a heart rate sensor configured to measure a fetal heart rate of at least one fetus carried by the given pregnant woman, the measured fetal heart rate and intensity of uterine contractions forming the cardiotocographic data;
a storage unit for a trained artificial intelligence model using a neural network, connected to a computing and display unit itself connected to the sensors,
the computing and display unit being configured to enable, subsequently to the training of the artificial intelligence model:
pre-processing cardiotocographic data of the given pregnant woman;
computing risk of fetal hypoxia at multiple points in time, based on processed cardiotocographic data measured at multiple points in time, using the trained artificial intelligence model,
wherein the artificial intelligence model is a neural network that has been trained in semi-supervised training mode:
a first, unannotated database containing only pre-processed first cardiotocographic training data obtained from pregnant women, with no fetal outcome data associated with these first cardiotocographic training data; and then
a second annotated database called “with fetal outcome”, containing: pre-processed second training cardiotocographic data obtained for these pregnant women, and fetal outcome data enabling the assessment of fetal hypoxia at birth associated with these second training cardiotocographic data.

2. The system according to claim 1, wherein the unannotated cardiotocographic data are derived from: records during labor, regardless of the number of hours before delivery, and/or records from pregnancy monitoring.

3. The system according to claim 1, wherein the processing blocks of the cardiotocographic data of the neural network are trained with the training cardiotocographic data of the first non-annotated database, so as to reproduce indicators characterizing the course of the heart rates of the fetus, these indicators being computed from the cardiotocographic data of the first training database.

4. The system according to claim 1, a wherein the processing blocks of the cardiotocographic data of the neural network are trained with the training cardiotocographic data of the first non-annotated database, so as to reproduce the cardiotocographic data of the first training database.

5. The system according to claim 1, wherein the processing blocks of the cardiotocographic data of the neural network are trained with the training cardiotocographic data of the first non-annotated database, so as to reproduce from a part of the input signal constituted by a part of the cardiotocographic data of the first pre-processed training database, a part of the masked input signal constituted by the cardiotocographic data of the first database.

6. The system according to claim 3, wherein the indicators are selected from the following list:
a baseline, computed as a sliding average of the fetal heart rate, excluding periods of acceleration and deceleration;
a duration and number of critical cardiac events of the acceleration type, each critical cardiac event of the acceleration type being determined by a fetal heart rate above a nominal heart rate range defined around the baseline;
a duration and number of critical cardiac events of the deceleration type, each critical cardiac event of the deceleration type being determined by a fetal heart rate below the nominal heart rate range defined around the baseline;
fetal heart rate variability, defined as the derivative of fetal heart rate on different time scales.

7. The system according to claim 1, wherein the second database presents maternal and/or fetal training clinical data, in addition to pre-processed second training cardiotocographic data and fetal outcome data, and wherein the computing and display unit being configured to enable, subsequently to the training of the artificial intelligence model with the clinical data:
the inputting or recording of maternal and/or fetal clinical data for the given pregnant woman, the clinical data being taken into account by the computing unit;
the computing of a risk of fetal hypoxia at multiple instants and from cardiotocographic data processed and measured at multiple instants, and clinical data of the given pregnant woman, using the trained artificial intelligence model.

8. The system according to claim 7, wherein the maternal and/or fetal training clinical data of the given pregnant woman and pregnant women for training, are selected from the following list:
maternal data relating to pregnant women, including age; and/or
fetal data relating to the fetus particularly comprising: gestational age and/or estimated fetal weight.

9. The system according to claim 7, wherein the maternal and/or fetal clinical data are selected from the following list:
maternal data on pregnant women, particularly comprising: body mass index, parity, indication of a singleton or twin pregnancy; and/or
fetal data relating to the fetus, particularly comprising sex; and/or
delivery data including, particularly comprising: suspected intrauterine infection through measurement of the pregnant woman's temperature, presence of vascular pathology such as gestational hypertension, pre-eclampsia, presence of gestational diabetes, cervical dilatation measurement.

10. The system according to claim 1, wherein:
the neural network is trained with the first unannotated database to obtain an encoder with blocks, making it possible to obtain column vectors of size M from vectors of dimensions, preferably with N/M greater than or equal to 10;
next, the neural network is trained with:
the fixed blocks of the encoder, applied to the second cardiotocographic data of the second database which are vectors of size and with the fetal outcome data, to obtain column vectors of size M, preferably with N/M greater than or equal to 10;
and advantageously, column vectors of size M are concatenated with column vectors of size C containing clinical data.

11. The system according to claim 3, wherein the computing and display unit is configured to, at the given display frequency:
compute, by applying a mathematical regression model, a statistical fetal hypoxia risk for the given woman; and
compute a contribution of this set of indicators to the statistical risk of fetal hypoxia, and simultaneously display the risk of fetal hypoxia computed by artificial intelligence and the contribution of these indicators to the statistical risk of fetal hypoxia,
wherein the mathematical regression model is of the type that makes it possible to determine a weighting coefficient for each parameter involved in computing the indicators and/or the risk of fetal hypoxia.

12. The system according to claim 11, wherein the mathematical regression model is a logistic regression model 24, wherein the fetal hypoxia risk RSK P is defined as
$$P = \frac{{exp}^{( {β}_{0} + {\sum }_{i = 1}^{n} {β}_{i} {x}_{i} )}}{1 + {exp}^{( {β}_{0} + {\sum }_{i = 1}^{n} {β}_{i} {x}_{i} )}} .$$
where:
the estimated risk of fetal hypoxia RSK, P, is a real number between 0 and 1, preferentially expressed as a percentage;
x1, . . . , xn correspond to indicators IND characterizing the course of the heart rates of the fetus,
the coefficients β are estimated on the retrospective database used to train the neural network.

13. The system according to claim 1, wherein the artificial intelligence model is trained from at least one of the following data representative of the fetal outcome:
a pH measurement at the umbilical cord of the fetus, with a pH value below 7.15 qualifying as moderate or severe fetal hypoxia, and a pH value below 7.05 qualifying as severe fetal hypoxia; and/or
gasometry measurements on the umbilical cord, in order, for example, to detect a base concentration greater than 12 mmol/L or a lactate concentration greater than 6 mmol/L; and/or
an Apgar score; and/or
possible hospitalization of the fetus in intensive care for suspected fetal hypoxia.

14. The system according to claim 1, wherein the artificial intelligence model is trained from cardiotocography recordings of pregnant women comprising:
a first, unannotated database, comprising the pre-childbirth records of pregnant women;
a second annotated database called “with fetal outcome”, comprising records located at most 40 minutes, advantageously at most 30 minutes, before the birth of the fetus carried by these pregnant women and annotated with fetal outcome data; and
advantageously, maternal and/or fetal clinical data from these women, whose fetal outcome is known.

15. The system according to claim 1, wherein the system comprises communication means between, on the one hand, the uterine contraction sensor and the heart rate sensor and, on the other hand, the computing and display unit, so that the system can be used on an outpatient basis or at home, the computing and/or display of the risk of fetal hypoxia being designed to be monitored remotely by a practitioner.

16. The system according to claim 1, wherein the cardiotocographic data processing blocks are a convolutional neural network or a transformer based on the attention mechanism, or a recurrent network, for example of the LSTM type.

17. The system according to claim 1, wherein the artificial intelligence model is a neural network that has been trained through semi-supervised training with, as input in addition to the training cardiotocographic data, the indicators computed therefrom.
